(19) United States
(12) Patent Application Publication
(10) Pub. No.: US 2004/0243076 A1
(43) Pub. Date: Dec. 2, 2004

(54) Hygiene product with a probiotic composition

(75) Inventors: Ulrika Husmark, Molnlycke (SE); Ingrid Gustafsson, Asa (SE)

Correspondence Address: BURNS DOANE SWECKER & MATHIS L L P, POST OFFICE BOX 1404, ALEXANDRIA, VA, 22313-1404, US

(73) Assignee: SCA Hygiene Products AB, Goteborg (SE)

(21) Appl. No.: 10/839,345

(22) Filed: May 6, 2004

Related U.S. Application Data
(60) Provisional application No. 60/469,832, filed on May 13, 2003.

(51) Int. Cl. A61F 13/20

(52) U.S. Cl. 604/358

(57) ABSTRACT

A hygiene product, such as a sanitary napkin, diaper, panty liner, tampon, incontinence guard, hygiene tissue and the like, includes a probiotic composition having a bacterial preparation of at least one lactic acid producing bacterial strain and a contact sorption drying carrier dispersed in a lipid phase. A method for producing a hygiene product with lactic acid producing bacteria, dried with the aid of contact sorption drying carriers, in a lipid phase is provided. The manufacturing process for the hygiene product has the advantages of economy, simplicity and bacterial survival during manufacturing and subsequent storage.

### CROSS-REFERENCE TO RELATED APPLICATION

This application claims the benefit of U.S. Provisional Application No. 60/469,832, entitled “Product,” filed on May 13, 2003, the entire contents of which are hereby incorporated by reference.

### BACKGROUND OF THE INVENTION

1. Field of the Invention

The present invention pertains to a hygiene product, such as a sanitary napkin, panty-liner, tampon, diaper, incontinence guard, hygiene tissue, etc., comprising a probiotic composition containing a dispersion of a bacterial preparation in a lipid phase. The bacterial preparation contains at least one lactic acid producing bacterial strain and at least one contact sorption drying carrier. The invention also pertains to a process for producing such a hygiene product.

2. Related Art

The urogenital area harbors a complex microbial ecosystem comprising more than 50 different bacterial species (Hill et al., Scand. J. Urol. Nephrol.86 (suppl.) 23-29 (1984)). The dominating species for fertile women in this area are lactic acid producing bacteria belonging to the genus Lactobacillus. These lactic acid producing members are important for retaining a healthy microbial flora in these areas, and act as probiotic bacteria with an antagonistic effect against pathogenic microbial species. Lactic acid producing bacteria inhibit growth and colonization by other microorganisms by occupying suitable niches for colonization, by forming biofilms and competing for available nutrients, thereby excluding colonization by harmful microorganisms. Additionally, the production of hydrogen peroxidase, specific inhibiting substances, such as bacteriocins, and organic acids (including lactic acid and acetic acid) that lower the pH, inhibit colonization by other microorganisms.

The microbial ecosystem of a healthy individual can be disturbed by the use of antibiotics, during hormonal changes, such as during pregnancy or use of contraceptives with estrogen, during menstruation, after menopause, in people suffering from diabetes, etc. Moreover, microorganisms may spread from the anus to the urogenital area, thereby causing infections. This results in a disturbance of the normal microbial flora and leaves the individual susceptible to microbial infections that cause vaginitis, urinary tract infections and ordinary skin infections. Microorganisms commonly associated with these kinds of infections belong to the genera Escherichia, Enterococcus, Psedomonas, Proteus, Klebsiella, Streptococcus, Staphylococcus, Gardnerella and Candida. Women are at particular risk due to their shorter distance between the anus and the urogenital tract. Young women are especially at risk because they do not yet have a well developed microflora in the urogenital area and older women, who no longer have a protective flora.

One way to reduce the problems with the kinds of infections described above is good personal hygiene. However, excessive use of cleaning agents not only decreases the amount of harmful microbes, but can harm the beneficial microbial flora, again rendering it susceptible for pathogenic species to colonize and cause infections. Alternatively, administration of lactic acid producing bacteria to the urogenital area and the skin in order to outcompete pathogenic species and facilitate reestablishment and maintenance of a beneficial microbial flora in these areas, has been found to be a successful means to treat and prevent microbial infections.

It has been suggested that lactic acid producing bacteria can be delivered via absorbent products, such as diapers, sanitary napkin, incontinence guards, panty liners and tampons, as described in, for example, in WO92/13577, WO97/02846, WO99/17813, WO99/45099 and WO00/35502.

A major concern with providing products intended to be used for transfer of lactic acid producing bacteria is that the bacteria have to retain viability during transport and storage of the products. Products comprising lactic acid producing bacteria are problematic in that the bacteria rapidly lose viability under moist conditions, and it is therefore important that the products are not exposed to moisture. One way to partly overcome this problem has been to supply products with freeze-dried lactic acid producing bacteria. However, if the bacteria in the products are not protected from moisture after manufacturing of the products, the air humidity will subsequently kill the bacteria and the shelf-life of such products will then be shortened. Another disadvantage with the direct application of dried lactic acid producing bacteria to a hygiene product, such as an absorbent product, is that transfer of the bacteria to the urogenital area will be low.

In order to overcome the problem with air humidity decreasing the shelf-life of products containing lactic acid producing bacteria it has been suggested to prepare dispersions of lactic acid producing bacteria and a hydrophobic substance, such as a fat or an oil. Research experiments have shown that storage in sterile vaseline oil results in a high level of viable lactobacilli cells after 8 months of storage (Arkadéva et al., NA. Nauchnye Doklady Vysshei Shkoly. Biologicheskie Nauki 2:101-104 (1983)). However, Stoianova et al. (Mikrobiologiia, 69:98-104 (2000)), found that immersion in mineral oil was not effective to preserve viability of lactic acid producing bacteria. U.S. Pat. No. 4,518,696 describes liquid suspensions of Lactobacilli in sunflower oil for oral administration to animals. However, none of the above references are concerned with the problems associated with retaining a high viability of lactic acid producing bacteria on hygiene products to be used to administer lactic acid producing bacteria to the urogenital area of a subject.

There are additional examples of the combination lactic acid producing bacteria and an oil, although these do not describe the effect of the oil on the survival of the lactic acid producing bacteria. WO01/13956 describes the use of pharmaceutical compositions comprising Emu oil, antimicrobial agents and/or Bacillus coagulans to be used for antimicrobial treatments. However, WO01/13956 does not describe how the problem with loss of viability during storage of the resulting products is to be solved. WO02/28446 described the use of an essentially hydrophobic carrier and freeze-dried lactic acid producing bacteria to prepare a distribution to be applied to an absorbent product. The hydrophobic carrier was mainly chosen to overcome problems with applying the bacteria to the absorbent product during manufacturing, but the carrier also protects the bacteria from air humidity.

As discussed above, products comprising lactic acid producing bacteria often contain freeze-dried bacteria since a high moisture content in the product result in products with shorter shelf-life due to reduced survival. Freeze-drying, however, is an expensive and complicated way of preparing bacteria with low moisture content. RU 2104299 describes an alternative method of drying bacteria, wherein the bacteria are mixed with contact sorption drying carrier before air-drying of the product. Nothing is disclosed in RU 2104299 about reducing the water content of the bacterial preparation by transferring the bacteria to a non-aqueous phase after the drying process.

In conclusion, there is still a need to develop products for delivery of lactic acid producing bacteria to urogenital area that are convenient to use, result in efficient transfer of the bacteria to the area where they are applied and that can be stored for long time periods without loss of viability of the bacterial cells. In addition, the manufacturing processes used to manufacture these products today are inefficient and expensive and there is a need to develop these to reduce manufacturing costs.

### OBJECTS AND SUMMARY

The above defined problems are solved in embodiments of the-present invention by using contact sorption drying carrier(s) for drying lactic acid producing bacteria, thereby improving the manufacturing process of hygiene products comprising lactic acid producing bacteria. Embodiments of the present invention provide for a hygiene product comprising a bacterial composition comprising lactic acid producing bacteria and contact sorption drying carrier(s) dispersed in a lipid phase. By using this approach, a hygiene product is obtained wherein the lactic acid producing bacteria are protected from moisture and which thereby has a prolonged shelf-life.

### BRIEF DESCRIPTION OF THE FIGURES

FIG. 1 depicts an illustrative example of an absorbent product, such as a sanitary napkin, diaper, panty liner, incontinence guard, and the like suitable for an embodiment of the present invention.

FIG. 2 shows a cross-section of the illustrative absorbent product depicted in FIG. 1 along the line II-II in FIG. 1.

FIG. 3 depicts a schematic illustration of a tampon comprising a probiotic composition according to an embodiment of the present invention.

FIG. 4 shows a cross-section of the illustrative absorbent product depicted in FIG. 3 along the line IV-IV in FIG. 3.

FIG. 5 shows the survival of bacterial cells in bacterial compositions according to an embodiment of the present invention during long term storage.

### DETAILED DESCRIPTION OF THE PREFERRED EMBODIMENTS

Definitions

By “contact sorption drying carriers” are meant substances that have the ability to take up moisture from the ambient environment. The contact sorption drying carriers as used in embodiments of the present invention, take up moisture from bacterial cells and bacterial microenvironments. Examples of contact sorption drying carriers suitable for embodiments of the present invention include, but are not limited to, oligo- and polysaccharides and inorganic agents. Further details of contact sorption drying carrier are given below.

By “hygiene product” is meant a hygiene product such as a sanitary napkin, incontinence guard, tampon, panty-liner, diaper, incontinence guard, hygiene tissue, etc.

By “probiotic composition” or “bacterial composition” is meant a composition comprising probiotic bacteria, i.e., bacteria that have the ability to reestablish the natural microbial flora of the host. The probiotic composition according to an embodiment of the present invention further comprises a lipid phase and contact sorption drying carrier(s).

By “dispersion” is meant a mixture of at least two phases.

By “preparation of at least one lactic acid producing bacterial strain,” “bacterial preparation,” “preparation of bacteria,” or “bacterial powder preparation” is meant a preparation comprising at least one lactic acid producing bacterial strain and at least one contact sorption drying carrier.

Preferred “lactic acid producing bacteria” suitable for embodiments of the present invention include, but are not limited to, bacteria from the genera Lactobacillus, Lactococcus and Pediococcus. Preferably the selected bacterium used is from the species Lactococcus lactis, Lactobacillus acidophilus, Lactobacillus curvatus or Lactobacillus plantarum. More preferably the bacterial strain is selected from Lactobacillus plantarum. Even more preferably the lactic acid producing bacterium is Lactobacillus plantarum 931 (deposition No. (DSMZ): 11918). The bacteria are preferably isolated from the natural flora of a healthy person, preferably the bacteria are isolated from the skin or urogenital area.

By “lipid phase” is meant a water-insoluble organic phase with a fatty character. Lipids suitable to be used in the lipid phase of embodiments of the invention include, but are not limited to, petroleum-derived lipids, synthetic lipids, and animal- and plant-derived lipids.

Examples of “additional components” include, but are not limited to, agents protecting the bacterial cells during drying of the bacteria, agents acting as nutrient for bacterial propagation, and skin caring agents. Further examples of suitable additional components are given below.

An object of the present invention is to provide hygiene products, such as sanitary napkins, tampons, panty-liners, diapers, incontinence guards, hygiene tissues, etc., suitable for absorbing bodily fluids and simultaneously delivering probiotic lactic acid producing bacteria to the skin, or more preferably, the urogenital area. The present invention pertains to solving the problems associated with providing products comprising lactic acid producing bacteria, such as problems with bacterial survival and costs and effectiveness of manufacturing. The prior art only discloses absorbent products comprising lactic acid producing bacteria in a lipid phase in which the bacteria have been freeze-dried. However, large scale freeze-drying of bacteria is not optimal since it is a complicated and expensive procedure.

The present inventors have found that by using alternative methods for drying the bacteria, a cheaper and simpler process for producing hygiene products comprising lactic acid producing bacteria can be provided. In this alternative process, a contact sorption drying carrier is used in the drying process in order to prepare a dried bacterial preparation that subsequently is mixed with a lipid phase and applied to a hygiene product.

The hydrophobic character of the lipid phase decreases the amount of air humidity which reaches the bacterial cells dispersed in the lipid phase, thereby increasing the survival time for the bacteria in the probiotic composition. Dispersing the lactic acid producing bacteria in a lipid phase has the additional advantage that transfer of the bacteria to the skin and/or urogenital area is enhanced compared to when no lipid phase is used. When the product is used, the lipid phase softens when exposed to body heat and the probiotic composition is transferred to the skin. When the bacteria come in contact with moisture after delivery to the skin, they are reactivated, start to grow and perform their probiotic action.

Contact sorption drying carriers suitable for embodiments of the present invention include, but are not limited to, oligo- and polysaccharides, such as starch, maltodextrin and beta-glucane, and inorganic agents, such as silicon dioxide (SiO₂). The amount of drying contact sorption carrier used is preferably between ca 10-50% by weight when added to a bacterial suspension before drying of the bacterial preparation. A too low amount of drying contact sorption carrier may result in too long drying times and a too high amount may make the resulting bacterial powder produced after drying hard to disperse in the lipid phase.

The water activity in the bacterial preparation comprising lactic acid producing bacteria and contact sorption drying carrier is preferably 0.30 or below, more preferably 0.25 or below, most preferably 0.20 or below. The amount of bacterial preparation in the lipid phase is preferably between 1-50% by weight, more preferably 5-25% by weight, most preferably 10-20%.

The bacterial preparation comprising lactic acid producing bacteria and contact sorption drying carrier is preferably a fine powder.

Lactic acid producing bacteria are chosen for the present invention due to their positive effect in preventing and treating microbial infection in the urogenital area and on the skin. The bacteria are preferably isolated from a healthy person, preferably from the skin or urogenital area of a healthy person. Preferred “lactic acid producing bacteria” for the object of the present invention include bacteria from the genera Lactobacillus, Lactococcus and Pediococcus. Preferably, the selected bacteria are from the species Lactococcus lactis, Lactobacillus acidophilus, Lactobacillus curvatus or Lactobacillus plantarum. More preferably, the selected bacterium is a Lactobacillus plantarum strain. Even more preferably, the lactic acid producing bacterium is Lactobacillus plantarum 931 (deposition No. (DSMZ): 11918). The lactic acid producing bacteria can be provided alone or in mixtures containing two or more bacterial strains.

The lipid phase used in embodiments of the present invention can be composed of a single lipid or a mixture of two or more lipids. Below a selection of lipids suitable for embodiments of the present invention is presented.

TABLE 1

| | | Melting | |
|---|---|---|---|
| Name | Producer | range | Main ingredients |
| Caremelt | Cognis¹⁾ | 25-58° C. | Triglyceride, paraffin, |
| 107 | | | monoglyceride |
| Caremelt 3 | Cognis | 30-47° C. | Triglyceride, paraffin, silicone |
| | | | wax, liquid triglyceride |
| Caremelt 58 | Cognis | 30-49° C. | Triglycerides, polymer wax, |
| | | | stearyl alcohol, silicone wax |
| Vaseline | AC Hud AB²⁾ | 5-45° C. | Petrolatum |
| Beeswax | Apoteket³⁾ | | Cera Flava |
| Akosoft 36 | Karlshamn⁴⁾ | 34-38° C. | Vegetable fat, hard fat |
| Lipex BC | Karlshamn | 35° C. | Hydrogenated Vegetable oil |
| AMS-C30 | DOW- | 70° C. | Silicone wax |
| | corning⁵⁾ | | |

¹⁾Henkel KgaA, Dusseldorf, Germany
²⁾Aco Hud AB, Stockholm, Sweden
³⁾Apoteket AB, Produktion och Laboratorier, Gothenburg, Sweden
⁴⁾Karlshamns AB, Karlshamn, Sweden
⁵⁾Seneffc, Belgium

According to embodiments of the present invention, the probiotic composition is applied on a hygiene product. For this purpose, it is desirable that the lipid phase has a melting behavior that allows the lipid phase to support bacterial survival on the hygiene product and not disturb the absorptive power of the hygiene product. A lipid phase with too low melting point tends to spread over the product and thereby reduces the absorptive power of the product. Also, a lipid phase with a low melting point tends not to enclose the bacteria to a high enough extent when the probiotic composition is spread on the hygiene product and thereby leaves a too large portion of the bacteria unprotected from air humidity, thereby reducing bacterial survival on the hygiene product. On the other hand, by choosing a lipid phase that has a melting behavior as specified by embodiments of the present invention, the lipid phase encloses the bacteria to a high enough extent. Thereby less of the bacterial population is exposed to atmospheric humidity, which results in a higher survival of the bacteria during storage of the hygiene product.

There is also an upper temperature limit for the melting behavior of the lipid phase. This limit is in part governed by the fact that for mixing the bacterial preparation with the lipid phase, the lipid phase is preferably soft enough in order to obtain a homogenous mixture. A lipid phase with too a high melting point has to be brought to temperatures that are too high for the bacteria to withstand while being mixed with the lipid phase and therefore too large a portion of the bacteria would die during preparation of the probiotic composition. Also, a lipid phase with too high a melting point may not be suitable for embodiments of the present invention because it does not soften to a high enough extent when in contact with the skin and therefore delivery of the bacteria to the skin is impaired.

The consistency of the probiotic composition is also influenced by the bacterial preparation which provide texture and consistency of the probiotic composition. By varying the ratio of amount of bacterial preparation to amount of lipid phase it is possible to achieve a bacterial composition with a suitable consistency for embodiments of the present invention.

Therefore, preferably, the lipid phase according to embodiments of the present invention is in major part solid at 30° C., becomes softer between 30° C. and 40° C., but is not fully melted at 50° C., preferably not fully melted at 60° C., most preferably not fully melted at 70° C. However, the lipid phase is still exhibiting soft properties from 30° C. This melting behavior can be achieved by using a single lipid or by mixing different lipids with different melting behaviors in order to achieve the desired melting behavior of the lipid phase. In order for the lipid phase not to interfere with bacterial survival due to a too high of a water content, the water content of the lipid phase preferably is 1% by weight or less, more preferably 0.1% by weight or less. The water vapor transmission rate of the lipid phase, measured according to ASTME 398-83 at 37.8° C. (100° F.) and 90% relative humidity (RH), is 10 g/m²/24 h or less, more preferably 5 g/m²/24 h or less, most preferably 2 g/m²/24 h or less.

Additional components can also be a part of the probiotic composition of the present invention or applied to a hygiene product of the present invention. Examples of such additional components include, but are not limited to, agents protecting the bacterial cells during drying of the bacteria, such as sugars (e.g., maltose, glucose, sucrose, trehalose, fructose), proteins (e.g., skim milk, albumin), amino acids (e.g., sodium glutamate), polyols (e.g., xylitol), mannitol and sorbitol, pH-regulating agents (e.g., lactic acid) and antioxidants (e.g., sodium ascorbate). Additional components also include nutrients that enhance bacterial propagation once the bacteria are activated by moisture after they are delivered to the skin or urogenital area. Examples of nutrients suitable for the present invention are sugars (such as maltose, glucose, sucrose, trehalose, fructose), polysaccharides (such as starch), vitamins (such as vitamin B and E) and proteins (such as skim milk). Suitable additional components also include skin caring substances, e.g., lipid soluble skin caring substances, such as vitamin A and E, skin caring oils, such as chamomile oils (Bisabolol), eucalyptus oil, lavender oil and phytosterols.

Briefly, in the process for producing the hygiene product comprising a probiotic composition of an embodiment of the present invention, a water suspension comprising at least one lactic acid producing bacterial strain (with or without additional agents protecting the bacterial cells during drying of the bacteria) is mixed with at least one dry contact sorption drying carrier. Immediately after addition of the contact sorption drying carrier, drying is spontaneously initiated. However to fulfill the drying, a drying step is carried out. After the drying step is carried out the dry bacterial preparation is dispersed in a lipid phase, which subsequently is added to the hygiene product.

In order to achieve a high survival rate during the drying step, the growth conditions for the bacterial cells have to be optimized in order to have bacteria with a fitness as high as possible during the drying step. The growth conditions, harvest procedures and the optional use of additional agents protecting the cells during the drying step, all affect the survival rate of the bacterial cells during drying and the subsequent storage of the probiotic composition. These conditions may be optimized for each bacterial strain. However, such an optimization is readily performed by a person skilled in the art. The concentration of bacterial cells in the bacterial water suspension to which the contact sorption drying carrier is added is 10⁶-10¹⁵ CFU (colony forming units)/ml, preferably 10¹⁰-10¹³ CFU/ml.

The drying step carried out after addition of contact sorption drying carrier to the bacterial water suspension can be performed by, e.g., convective drying methods, contact drying methods or by using electromagnetic radiation. Examples of convective drying methods suitable for the present invention include, but are not limited to, spray drying, spray granulation and fluidized bed drying. The common feature for convective drying methods is that warm and dry gas flushes around the product and enters into a heat and mass transfer with the product. Convective methods transfer required heat and/or dryness by convection to the wet product. During contact drying, the wet product is stationary in touch with a warm surface or constantly brought into new contact with the warm surface by stirring or revolving. Drying by electromagnetic radiation (infrared or microwave radiation) involves using a belt dryer or a stationary support and submitting the wet product to electromagnetic radiation energy which is being absorbed by the wet product. The absorbed energy serves to warm up the product whereby the moisture in the wet product is evaporated. Drying times using electromagnetic radiation often result in very short drying times. Before the drying step additional components, such as nutrients and protecting agents, can be added to the bacterial cells. Examples of such additional components are given above.

A probiotic composition is thereafter prepared wherein the dry bacterial preparation, comprising lactic acid producing bacteria and contact sorption drying carrier, is dispersed in a lipid phase. During this step, further additional components, such as nutrients for bacterial propagation and skin caring substances can be added to the probiotic composition.

The probiotic composition is finally added to the hygiene product. Preferably, the probiotic composition is applied in a stripe(s) or patch(es), since covering a too large part of the hygiene products would result in reduced absorptive properties of the product. Preferably, not more than 40% of the surface of the hygiene product is covered by the probiotic composition. More preferably, not more the 20% of the surface of the hygiene product is covered by the probiotic composition.

The present invention also relates to hygiene products comprising a probiotic composition, produced by the process described above.

The probiotic composition according to the present invention is applied to a hygiene product, such as a hygiene tissue, incontinence guard, diaper, panty liner, tampon, sanitary napkin, etc.

By “hygiene tissue” is meant any device for wiping skin, for instance, a washcloth, patch, towelette, napkin, wetwipe, and the like. The hygiene tissue provided can be composed of a matrix comprising any natural or synthetic fiber, such as rayon, cellulose, regenerated cellulose, polyester, polyolefine fibers, textile and the like, or foam, nonwoven, felt or batting, or combinations thereof. The probiotic composition according to embodiments of the present invention is applied or impregnated to the hygiene tissue matrix.

The probiotic composition of embodiments of the present invention, is, as described above, particularly suitable for application to absorbent products, such as sanitary napkins, incontinence guards, panty-liners, diapers, tampons, etc, since these products provide a convenient means for delivery of lactic acid bacteria to the urogenital area. The sanitary napkins, incontinence guards, panty-liners, tampons, and diapers according to an embodiment of the invention preferably comprise of a liquid permeable casing sheet facing the user and an absorbent layer comprised of one or more layers, placed beneath or inside the liquid permeable casing sheet. The probiotic composition is placed onto the casing sheet or inside it.

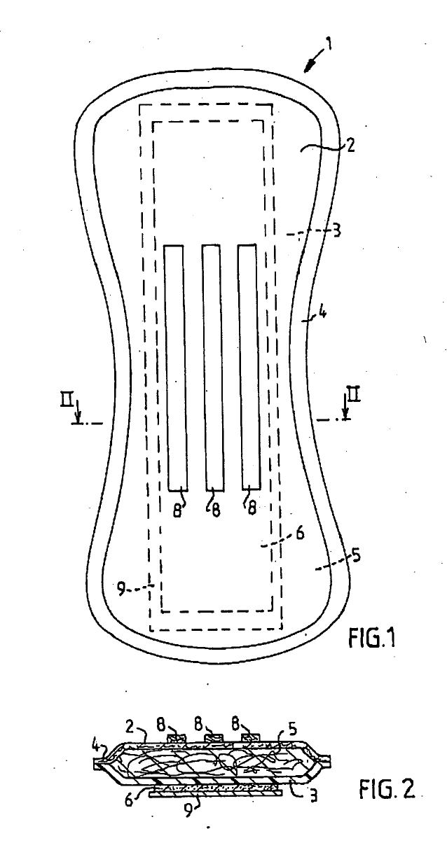

Below a more detailed description of an absorbent product, such as a sanitary napkin, panty liner, diaper or incontinence guard is given. The absorbent product 1 shown in FIG. 1 and FIG. 2 (cross-section of the absorbent product depicted in FIG. 1 along the line II-II in FIG. 1) includes a liquid-permeable casing sheet or top sheet 2 disposed on that side of the absorbent product which is intended to lie proximal to the wearer in use. The liquid-permeable casing sheet 2 will conveniently consist in a somewhat soft, skin-friendly material. Different types of non-woven material are examples of suitable liquid-permeable materials. Other casing sheet materials that can be used are perforated plastic films, net, knitted, crocheted or woven textiles, and combinations and laminates of the aforesaid types of material.

The absorbent product 1 also includes a liquid-impermeable casing sheet or backing sheet 3, disposed on that side of the napkin 1 distal from the wearer in use. The liquid-impermeable casing sheet 3 is conventionally comprised of thin plastic film. Alternatively, there may be used a liquid-permeable material that has been rendered impermeable to liquid in some way or another. For instance, the liquid-permeable material may be coated with a glue that is impermeable to liquid, and the liquid-permeable layer laminated with a liquid-impermeable material, or hot-calendering a material that was initially liquid-permeable, such as to melt down the surface of the material and therewith obtain a liquid-impermeable layer. Alternatively, there may be used other textiles comprised of hydrophobic fibers and so impervious as to enable them to be used as a liquid barrier layer. The liquid-impermeable casing sheet 3 may beneficially be vapor permeable.

The two casing sheets 2, 3 form a joining edge 4 that projects outwardly around the napkin contour line, and are mutually joined at this edge. The sheets may be joined together by means of any appropriate conventional technique, such as gluing, welding or sewing.

The absorption core 5 sandwiched between the casing sheets 2, 3 may constitute the layer capable of receiving and storing essentially all liquid discharged by the wearer. The absorption core 5 may, for instance, be produced from cellulose pulp. This pulp may exist in rolls, bales or sheets that are dry-defibered and converted in a fluffed state to a pulp mat, sometimes with an admixture of superabsorbents, which are polymers capable of absorbing several times their own weight of water or body liquid (fluid). Examples of other usable materials are different types of foamed materials known, for instance, from SE 9903070-2, natural fibers, such as cotton fibers, peat, or the like. It is, of course, also possible to use absorbent synthetic fibers, or mixtures of natural fibers and synthetic fibers. Patent Application SE 9903070-2 describes a compressed foam material of regenerated cellulose, e.g., viscose. Such foam material will preferably have a density of 0.1 to 2.0 g/cm³. The absorbent material may also contain other components, such as foam-stabilizing means, liquid-dispersing means, or a binder, such as thermoplastic fibers, for instance, which have been heat-treated to hold short fibers and particles together so as to form a coherent unit.

A fastener means 6 in the form of an elongate rectangular region of self-adhesive is provided on the surface of the liquid-impermeable casing sheet 3 that lies distal from the wearer in use. The fastener means 6 extends over the major part of the liquid-impermeable casing sheet 3. The invention is not restricted to the extension of the fastener means 6, and said means may have the form of elongate stripes, transverse regions, dots, circles, or other patterns and configurations. Neither is the invention restricted to the use of solely adhesive fastener means, since friction fasteners may be used and other types of mechanical fasteners, such as press studs, clips, girdles, pants or the like may be used when found suitable to do so. When an adhesive fastener means is used this is commonly protected, by a protective layer 9, from adhering to other surfaces prior use, which would destroy the fastener means.

In FIGS. 1 and 2 one way to place the probiotic composition is exemplified, wherein the probiotic composition is placed in stripes 8.

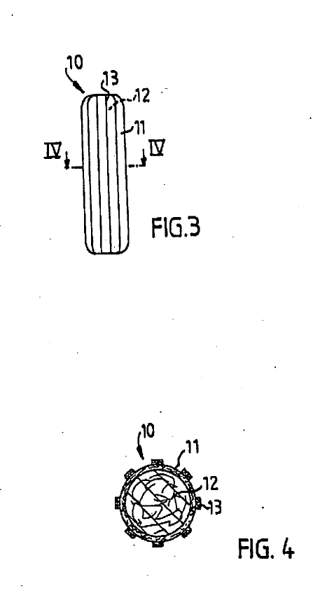

In a similar manner to what is described above, a tampon comprising the probiotic composition can be prepared. FIGS. 3 and 4 (cross-section of the tampon in FIG. 3 along the line IV-IV) depict a schematic exemplary drawing of a tampon 10 comprising a probiotic composition according to the present invention, wherein the probiotic composition 13 is arranged onto the casing sheet 11 in stripes. Also depicted is the absorbent core 12.

The skilled person could easily use the above exemplary descriptions of the hygiene products described above which comprise a probiotic composition according to embodiments of the present invention, to manufacture a tampon, sanitary napkin or any other hygiene product comprising a probiotic composition according to an embodiment of the invention. Therefore, alternative designs of a sanitary napkin, incontinence guard, panty-liner, diaper, tampon, hygiene tissue, etc., are also included in the present invention.

In one preferred embodiment of the present invention, a lipid phase (which comprises or does not comprise lactic acid producing bacteria) which melts at a higher temperature is placed underneath the probiotic composition. The lower lipid phase then functions as a protecting layer that inhibit the probiotic composition to be spread over the hygiene product when the probiotic composition melts. Thereby, the risk of inhibition of absorption by the lipid phase is even more decreased, since the lower lipid phase does not melt, or melts to a lower extent, than the upper phase, when in contact with body heat or during storage at elevated temperatures.

In addition to enhancing bacterial survival, the use of a lipid phase in which the lactic acid producing bacteria are dispersed also enhances transfer rates of the probiotic composition to the skin and urogenital area. This can be an effect of the lipid having more “adhesive” properties than, for example, water, thereby resulting in a higher amount of bacteria actually being transferred to the skin. Also, the lipid has the effect of enhancing bacterial survival once the bacteria are delivered to the skin, presumably because the lipid creates a micromilieu, that is beneficial for retaining bacterial viability and that enhances growth of the bacteria once added to the skin.

The positive effect of embodiments of the present invention lies in advantages associated with the manufacturing of products comprising lactic acid producing bacteria (since the present invention solves many of the problems associated with the production of such products). The use of contact sorption drying carriers may improve the survival rate of the bacterial cells during the drying step, probably by stabilizing the cell membrane. In addition, the use of contact sorption drying carriers can allow a fine powder of dried bacteria to be achieved directly (a fine powder is necessary if the dried bacteria are to be mixed with a lipid phase). If the dried bacteria achieved are not in the form of a fine powder, which is the case after the commonly used freeze-drying, a further step of grinding or sifting is necessary to produce the fine powder. This adds stress to the bacteria, and, during the extra time required to perform the grinding or sifting the bacteria may take up moisture from the environment, which might adversely affect bacterial survival. In comparison, by using a contact sorption carrier according to the present invention a fine powder can be achieved directly, thereby avoiding the grinding/sifting step. The use of a contact sorption drying carriers can also speed up the drying process and aid in reaching a lower water activity, which is an advantage in terms of bacterial survival and economy. Also, the use of contact sorption drying carriers can provide a more economical means for drying bacterial cells, since sublimation of water (as in freeze-drying) is more energy consuming than evaporation, which is used in the present invention. In addition the investment costs for a freeze-drying plant are high. It can also be difficult to apply freeze-drying in a continuous process, and such a process can be advantageous when the production of large amounts of dry bacteria is necessary.

Therefore, in conclusion, by producing the hygiene products comprising probiotic composition according to the present invention, several advantages in terms of economy, simplicity in manufacturing and bacterial survival during manufacturing and during subsequent storage, can be achieved.

The present invention will now be described by the use of illustrative examples that are not intended to be limiting of the present invention.

### EXAMPLES

### Example 1

Procedure for Drying with SiO₂ as Contact Sorption Drying Carrier and Convective Drying:

An aqueous suspension of L. plantarum 931 (deposition No. (DSMZ): 11918) in 10% Na-glutamate and 10% glucose was used as a starting material. The cell concentration was 6.5×10¹¹ cfu/ml. 80 ml of the suspension was mixed with 160 ml of SiO₂ (Aerosil 200, Degussa Norden AB, Malmö, Sweden). Drying was performed in box with circulated dry air (33° C., 0.8% relative humidity). The bacterial powder preparation was dry after approx. 6-8 hours. The final amount of bacterial preparation was 27 g with 2.4×10¹¹ cfu/g, which corresponds to a calculated survival rate of 12%. The water activity of the powder was 0.034 measured with an equipment from Aqualab, model 3TE (Decagon Devices Inc., Pullman, Wash., USA).

### Example 2

Comparison of Convective Drying and IR-drying with and without Different Contact Sorption Drying Carriers

The bacteria (L. plantarum 931 (deposition No. (DSMZ): 11918)) were suspended in deionized water or in an aqueous solution of 10% trehalose. The contact sorption drying carriers tested were aerosil 200 (Degussa Norden AB, Malmö, Sweden), native potato starch and β-glucane. The cell concentration was 10¹³-10¹⁴ cfu/ml. 2.5 g aerosil was added to 20 ml of cell suspension, 30 g β-glucane was added to 25 ml cell suspension and 27 g potato-starch was added to 25 ml cell suspension, in order to achieve a “porridge” with a consistency convenient to handle. The final drying of the bacterial preparation to a powder was performed either via convective drying (40° C., for maximum 3 hours) or with fast drying with IR (infrared light, 40-58° C. for 5-7 minutes).

The results of the experiment are presented in Table 2. A very satisfactory survival rate of the bacteria was achieved. The addition of trehalose did not affect the survival rate of the bacteria. Addition of sugars during drying of bacterial cells is commonly considered necessary. However, in the drying process of the present invention, this was not necessary. Both drying methods, convective drying and IR drying, respectively, gave similar results in terms of survival rates. The use of aerosil or potato starch resulted in the best powder structure (i.e., a more fine powder). All the used contact sorption drying carriers resulted in high survival rates.

TABLE 2

| | Drying | Drying | | CFU/g | |
|---|---|---|---|---|---|
| Sugar | carrier | method | Final A_{w} | powder | % survival |
| Trehalose | Aerosil | IR | 0.19 | 2.2E13 | 3.9 |
| — | Aerosil | IR | 0.26 | 2.7E13 | 4.7 |
| Trehalose | β-glucan | Convective | 0.19 | 5.2E12 | 20.8 |
| — | β-glucan | Convective | 0.20 | 5.4E12 | 54.2 |
| Trehalose | β-glucan | IR | 0.26 | 1.0E12 | 3.9 |
| — | β-glucan | IR | 0.24 | 2.5E12 | 27.2 |
| Trehalose | Pot. starch | Convective | 0.16 | 1.0E14 | 83 |
| — | Pot. starch | Convective | 0.17 | 5.0E14 | 100 |
| Trehalose | Pot. starch | IR | 0.22 | 4.0E14 | 100 |
| — | Pot. starch | IR | 0.20 | 1.0E13 | 52 |

### Example 3

Comparison of Drying Efficacy with and without Contact Sorption Drying Carrier

A water suspension of L. plantarum 931 (deposition No. (DSMZ): 11918) with a concentration of 5.7×10¹¹ cfu/ml was mixed with the contact sorption drying carriers, aerosil (Degussa) and potato starch, respectively, and dried in a convective drying chamber (34° C. and 1% relative humidity) for 24 hours. The water activity was determined after the drying (i.e. after 24 hours).

The results of Example 3 are presented in Table 3. As can be seen in Table 3, the water activity was one order of magnitude higher when no contact sorption drying agent was used. The water activity according to the present invention is preferably 0.30 or below and such low water activity levels could not be obtained without the use of a contact sorption drying carrier according to the present invention.

TABLE 3

| | Drying | A_{w} in |
|---|---|---|
| Sugar | carrier | powder |
| Trehalose | None | 0.45 |
| 10% | | |
| Trehalose | Aerosil | 0.038 |
| 10% | | |
| Trehalose | Pot. starch | 0.027 |
| 10% | | |
| Sorbitol | None | 0.258 |
| 10% | | |
| Sorbitol | Aerosil | 0.041 |
| 10% | | |
| Sorbitol | Pot. starch | 0.022 |
| 10% | | |

### Example 4

Convective Spray-drying

A water suspension of L. plantarum 931 (deposition No. (DSMZ): 11918) was mixed with 10% maltodextrine and spray-dried at two different air temperatures. The feed-flow was 5 ml/minute and the air-flow was 800.1/minute. The resulting powder was very fine (grain size approximately 5-10 μm). The survival rates of this experiment are presented in Table 4 below.

TABLE 4

| | Temp in/ | | Cfu/g | |
|---|---|---|---|---|
| Contact sorption drying agent | Temp out | A_{w} | powder | % survival |
| Maltodextrine | 180/90 | 0.12 | 2.5E10 | 17 |
| Maltodextrine | 150/75 | 0.16 | 9.6E10 | 61 |

### Example 5

Survival in a Bacterial Composition During Long Term Storage.

A bacterial powder preparation was produced with the process described in Example 1. The bacterial powder was dispersed in different lipid phases (beeswax, Caremelt 58, Caremelt107, or vaseline) directly after production by melting the lipid phase and dispersing the powder in the lipid phase (1 g powder to 9 g lipid phase). The resulting bacterial composition was poured into open glass vials and allowed to solidify. The vials were very loosely covered with a aluminum-foil and placed in a standard climate chamber (23° C., 50% relative humidity). At certain times (during a time period of 8 months) the number of surviving cells was measured. For sampling, 1 g of bacterial composition was scraped from the surface, placed in 9 ml of NaCl, stomached for 1 minute and the number of CFU was measured with spread-plate technique on MRS-agar.

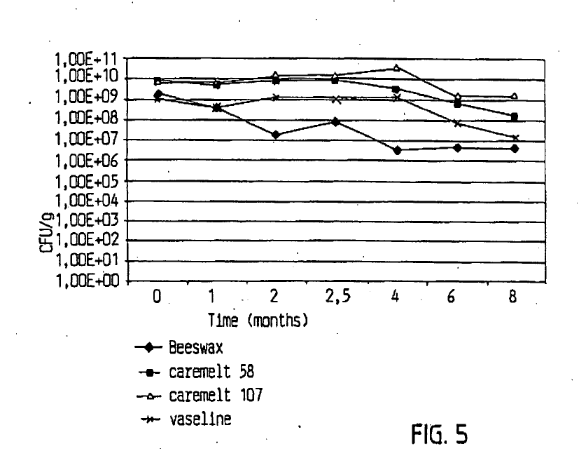

The very high survival rates obtained are presented in FIG. 5.

### Example 6

Transfer of a Bacterial Composition of the Present Invention from a Panty-liner Surface to Skin.

The bacterial powder preparation was produced with the process described in example 1. The powder was dispersed directly after the production in different melted waxes (1 g powder to 9 g wax). With a pilot scale printer the produced bacterial composition was printed to the surface layer of panty-liners. The printed pattern was dots (ca 3 mm in diameter, distance ca 3 mm)

The transfer test was performed with specimens, a circle of 2.5 cm in diameter, punched out from the products. 10 μl of NaCl was added to the product with a pipette, and the specimen subsequently mounted, with constant pressure (elastic tape, and elastic bandage), on to the forearm of volunteers. After 2 hours, the product was removed and the number of lactobacilli on the skin measured. A sterile stainless-steel cylinder (2.6 cm in diameter, height 2 cm) was held tight to the skin at the site that had been covered with the specimen, and 1 ml of phosphate buffer (0.1M, pH 7.2) was poured into the cylinder. With a smooth glass-stick the skin was gently “kneaded” for 1 minute. Afterwards, the buffer was collected with a pipette and the CFU measured with pour plate technique and on MRS-agar. The amount of indigenous lactic acid producing bacteria in this place of the body is very low compared to the amount of bacteria transferred and therefore all the counted bacteria were taken as transferred bacteria.

The percentage of bacterial cells in the bacterial composition was calculated as follows:

X=number of CFU/specimen before transfer test

Y=number of transferred CFU

Percentage transfer=Y/X.

As a control, a specimen wherein the bacteria were not mixed with a lipid phase before placement on the panty-liner was used. Instead, as a control specimen, a panty-liner to which a bacterial suspension (without contact sorption drying carrier) had been applied whereafter the panty-liner with the applied bacteria had been dried, was used.

TABLE 5

| Lipid phase | Percentage transfer |
|---|---|
| Carmelt 3 | 22.8 |
| Caremelt 58 | 47.9 |
| Vaseline | 89.6 |
| Beeswax | 27.0 |
| Control | 2.13 |

As can be seen in Table 5, the amount of bacteria transferred to the skin from the panty-liner provided with the probiotic composition of an embodiment of the present invention results in very high transfer rates of bacterial cells from the product to the skin.

Although only preferred embodiments are specifically illustrated and described herein, it will be appreciated that many modifications and variations of the present invention are possible in light of the above teachings and within the purview of the appended claims without departing from the spirit and intended scope of the invention.

What is claimed is:

1. A hygiene product comprising a probiotic composition comprising a dispersion of a bacterial preparation in a lipid phase, said bacterial preparation containing at least one lactic acid producing bacterial strain and at least one contact sorption drying carrier.

2. The hygiene product of claim 1, wherein the lactic acid producing bacterium is isolated from the skin or urogenital area of a healthy person.

3. The hygiene product of claim 1, wherein the lactic acid producing bacterial strain is selected from the genera Pediococcus, Lactococcus, Lactobacillus or a mixture thereof.

4. The hygiene product of claim 3, wherein the lactic acid producing bacterial strain is Lactobacillus plantarum.

5. The hygiene product of claim 4, wherein the lactic acid producing bacterial strain is Lactobacillus plantarum 931 (deposition No. (DSMZ): 11918).

6. The hygiene product of claim 1, further comprising at least one additional component selected from the group consisting of agents protecting the cell during drying of the bacteria, agents acting as nutrients for bacterial propagation, and skin caring substances.

7. The hygiene product of claim 1, wherein the water activity of the bacterial preparation is 0.30 or below.

8. The hygiene product of claim 7, wherein the water activity is 0.25 or below.

9. The hygiene product of claim 8, wherein the water activity is 0.20 or below.

10. The hygiene product of claim 1, wherein the water content of the lipid phase is 1% by weight or less.

11. The hygiene product of claim 10, wherein the water content is 0.1% by weight or less.

12. The hygiene product of claim 1, wherein the water vapor transmission rate of the lipid phase, measured according to ASTME 398-83 at 37.8° C. (100° F.) and 90% relative humidity (RH), is 10 g/m²/24 h or less.

13. The hygiene product of claim 12, wherein the water vapor transmission rate is 5 g/m²/24 h or less.

14. The hygiene product of claim 13, wherein the water vapor transmission rate is 2 g/m²/24 h or less.

15. The hygiene product of claim 1, wherein the hygiene product is a sanitary napkin, a panty-liner, a tampon, a diaper, an incontinence guard, or hygiene tissue.

16. A process for producing a hygiene product comprising a dispersion of a preparation of a least one lactic acid producing bacterial strain and at least one contact sorption drying carrier in a lipid phase comprising:
a) mixing at least one lactic acid producing bacterial strain with at least one contact sorption drying carrier;
b) drying the bacterial preparation comprising the at least one lactic acid producing bacterial strain and the at least one contact sorption drying carrier;
c) dispersing the bacterial preparation in the lipid phase, thereby producing a probiotic composition;
d) applying the probiotic composition to the hygiene product.

17. The process of claim 16, wherein the concentration of the bacterial strain mixed with the at least one contact sorption drying carrier is 10⁶-10¹⁵ colony forming units/ml.

18. The process of claim 16, wherein the drying is carried out by a convective drying method, a contact drying method, or by electromagnetic radiation.

19. The process of claim 16, wherein at least one additional component is added when the bacterial preparation is dispersed in the lipid phase and wherein the at least one additional component is selected from the group consisting of agents protecting the cell during drying of the bacteria, agents acting as nutrients for bacterial propagation, and skin caring substances.

20. The process of claim 16, wherein the probiotic composition is applied to not more than 40% of the surface of the hygiene product.
